(19) United States
(12) Patent Application Publication
(10) Pub. No.: US 2016/0175094 A1
(43) Pub. Date: Jun. 23, 2016

(54) Morphological sewing cuff assembly for heart valve

(71) Applicant: Howard Song, Portland, OR (US)

(72) Inventor: Howard Song, Portland, OR (US)

(21) Appl. No.: 14/757,955

(22) Filed: Dec. 23, 2015

Related U.S. Application Data
(60) Provisional application No. 62/096,144, filed on Dec. 23, 2014.

(51) Int. Cl. A61F 2/24

(52) U.S. Cl. CPC A61F 2/246; A61F 2/2442

(57) ABSTRACT

An embodiment includes a prosthetic heart valve comprising: a supraannular aortic valve body having a longitudinal axis and outlet and inlet ends; a leaflet in the valve body to allow blood flow from the inlet end to the outlet end; and a sewing cuff, surrounding the valve body, having upper and lower portions; wherein (a)(i) the first cuff includes extends below the inlet end but does not extend above the outlet end; (a)(ii) the lower portion slopes down and away from the long axis; (c) the lower portion includes scalloped portions; wherein the lower portion includes first and second voids separating the lower portion into at least first, second, and third portions that each rotate vertically independently of each other. Other embodiments are described herein.

### CROSS-REFERENCE TO RELATED APPLICATION

This application claims priority to U.S. Provisional Patent Application No. 62/096,144 filed on Dec. 23, 2014 and entitled “Morphological Sewing Cuff Assembly for Heart Valve”, the content of which is hereby incorporated by reference.

### BACKGROUND

Historically, mechanical heart valve prostheses have been constructed with continuous cylindrical sewing cuff rings. When considering the operative approach to implanting the prosthesis, such a design restricts the surgeon to placing attachment sutures in roughly a 2-dimensional plane. This 2-dimensional attachment methodology generally “flattens” the annulus and could potentially change the hemodynamic flow of blood upon entering/exiting the prosthetic valve.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1-5 include various views of an embodiment.

FIG. 6 includes a method of implantation in an embodiment.

### DETAILED DESCRIPTION

In the following description, numerous specific details are set forth. However, it is understood that embodiments of the invention may be practiced without these specific details. Well-known structures and techniques have not been shown in detail to avoid obscuring an understanding of this description. References to “one embodiment”, “an embodiment”, “example embodiment”, “various embodiments” and the like indicate the embodiment(s) so described may include particular features, structures, or characteristics, but not every embodiment necessarily includes the particular features, structures, or characteristics. Further, some embodiments may have some, all, or none of the features described for other embodiments. Also, as used herein “first”, “second”, “third” describe a common object and indicate that different instances of like objects are being referred to. Such adjectives are not intended to imply the objects so described must be in a given sequence, either temporally, spatially, in ranking, or in any other manner. Also, the terms “coupled” and “connected,” along with their derivatives, may be used. In particular embodiments, “connected” may be used to indicate that two or more elements are in direct physical contact with each other and “coupled” may mean that two or more elements co-operate or interact with each other, but they may or may not be in direct physical or electrical contact. Also, while similar or same numbers may be used to designate same or similar parts in different figures, doing so does not mean all figures including similar or same numbers constitute a single or same embodiment.

A goal of an embodiment is to provide a flexible sewing cuff ring for aortic mechanical heart valves that better approximates native anatomy. The embodiment allows for a better match with actual anatomy without distorting (or minimally distorting) the 3-dimensional structure of the aortic annulus. The embodiment allows for a reduction of stresses associated with the “flattening” of the annulus typically associated with conventional devices, and therefore causes less anatomical distortion, which minimizes suture “tear-through” and reduces the potential for paravalvular leak. In addition to clinical improvements, implantation of a mechanical heart valve with the embodiment of a “morphological” sewing cuff ring allows for greater ease of implantation since native anatomy is not altered (“flattened”) in the process.

Embodiments are shown in the figures described herein. In an embodiment, the flexible shape of the cuff allows the surgeon to deploy attachment sutures three-dimensionally for improved implantation. This contrasts with traditional two-dimensional sewing cuffs that restrict placement to a thin two-dimensional plane of attachment, thus, “flattening” the native annulus. For example, the flexible nature of the cuff allows for an improved mating of sewing cuff attachment points to native aortic anatomy through the use of traditional sutures.

In an embodiment the cuff is an integral part of a replacement valve, such as mechanical aortic heart valve prosthesis.

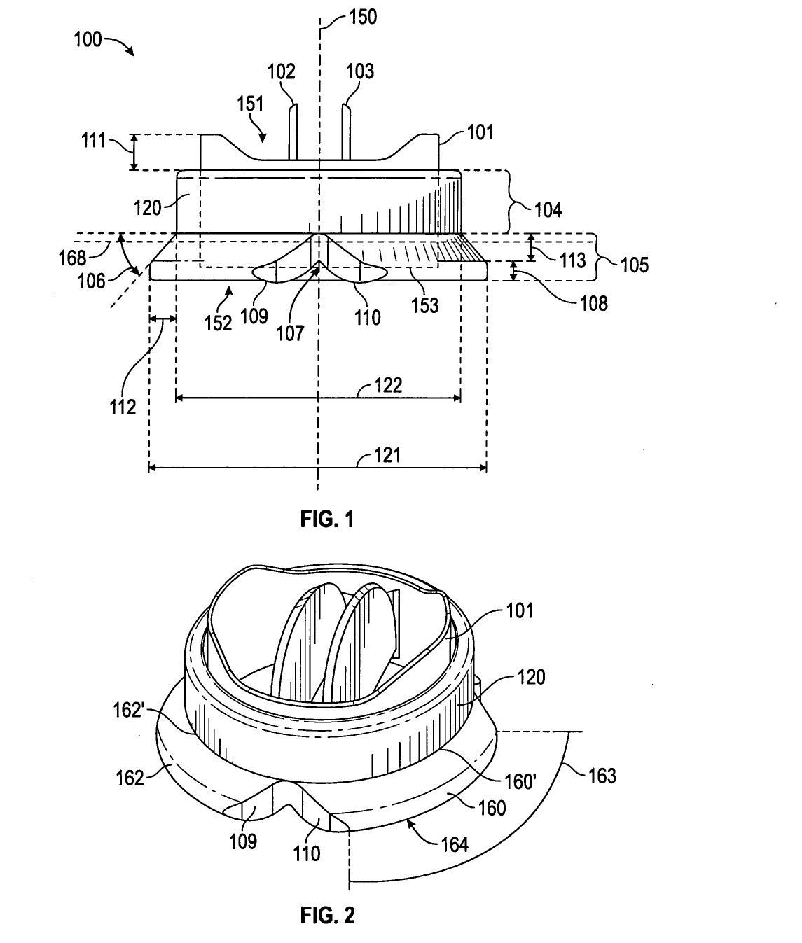

FIG. 1 includes a front view of a morphological sewing cuff ring in an embodiment. FIGS. 2-5 include isometric views of a morphological sewing cuff ring in an embodiment.

The embodiment of FIG. 1 includes a system 100 with a tubular valve body 101 having longitudinal axis 150, an outlet end 151 (which is the “downstream” end of the device), an inlet end 152 (which is the “upstream” end of the device), an inner surface, and an outer surface. The outer surface comprises sewing cuff 120. Two leaflets 102, 103 are shown (in their open position) within the tubular valve body. The leaflets intermittently and reversibly seal across the valve body inner surface to allow unidirectional flow (i.e., substantially unidirectional flow but not necessarily absolute unidirectional flow) through the tubular valve body from the inlet end to the outlet end. Embodiments are not limited to any single number of leaflets.

In an embodiment cuff 120 extends below inlet end 152 of the valve. Specifically, the inlet end has a bottom most edge 153 and cuff 120 extends below this edge by a distance 108. Lower cuff portion 105 has an upper subportion that overlaps body 101 a distance 113 and another subportion that extends below body 101 a distance 108. Element 107 shows a portion of edge 153 that is visible between adjoining scalloped portions 109, 110. This is advantageous because the contour of the free edge of the cuff corresponds more closely to the natural contour of the aortic valve annulus to which it will be sewn. It therefore can be implanted with less stress and distortion of the aortic valve annulus. Because less stress is applied to the aortic valve annulus, a larger size valve can be implanted. Larger valves are favorable to the patient because they offer less resistance to blood flow passing through the valve.

In an embodiment portion 104 does not extend to the top of body 101 and instead is offset from the top of portion 101 by a distance 111.

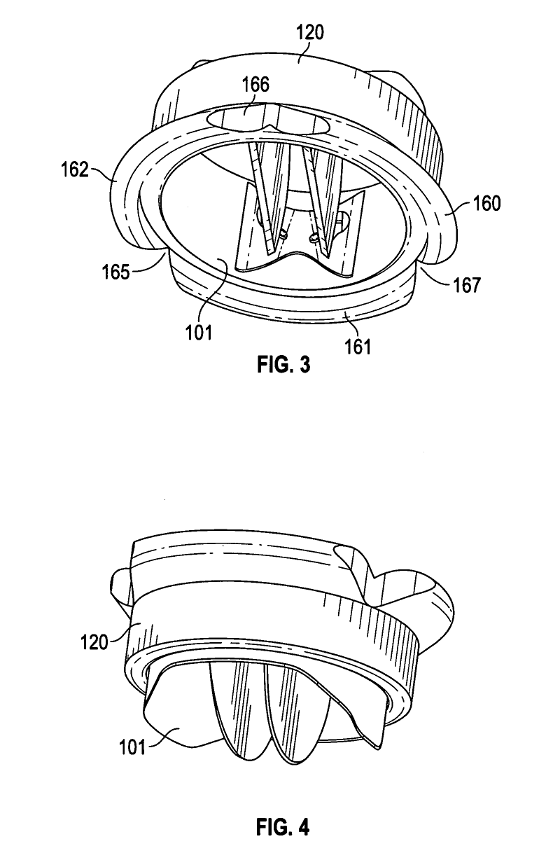

Scalloped portions 109, 110 (also referred to as portions 162, 160 in FIG. 3) are shown as being divided by central vertical axis 150, but in other embodiments the sewing cuff's scalloped portions may be rotated with respect to a central axis 150 that also divides leaflets 102, 103. For example, portions 109, 110 may be rotated 45 degrees to the left or right in FIG. 1 such that no scalloped portions align directly between leaflets 102, 103 as is shown in FIG. 1.

As seen in FIG. 3, there are multiple portions of portion 105 that include scalloping. For example, in FIG. 3 three such locations are shown which provide flexibility to portion to 105 so the surgeon can better manipulate the ring to match patient anatomy. Further, the three portions better align with fibrous crown shaped portions of the annulus (see FIG. 6). The native aortic valve annulus does not correspond to a flat circle. That is, it is a three dimensional structure with three peaked portions, similar to the top edge of a crown. As seen in FIG. 6, an embodiment of the sewing cuff has been modified so its lower portion has three peaked portions, corresponding to the natural contour of the aortic valve annulus to which it is attached at the time of implantation.

In an embodiment cuff 120 is formed from two subparts that may be independent from one another. For example, portion 105 may be coupled to portion 104 via stiches, adhesives, resistance fit, and the like. In an embodiment portion 105 fits between projections on cuff 120 and/or body 101 to provide retention of portion 105 via resistance fit. However, the resistance fit prevents movement of portion 105 along the vertical direction (parallel to axis 150) but does allow for rotation of portion 105 about axis 150. Both portions 104 and 105 may rotate about body 101 in some embodiments. Allowing rotation of portion 105 allows a surgeon to better align scalloped portions 109, 110 with anatomical elements, such as fibrous tissue portions of the heart.

In an embodiment portion 104 extends all the way to edge 153 with portion 105 formed along the outside of portion 104. In other embodiments portion 105 merely abuts portion 105 with each having equivalent minimum diameters and each directly contacting an outer surface of body 101.

In an embodiment lower portion 105 is wider than portion 104. Lower portion 105 may have a maximum diameter 121 that is greater than a maximum diameter 122 of upper portion 104. Along one portion of the cuff, lower portion 105 extends a distance 112 beyond that of upper portion 104. This is advantageous because the distance 112 allows a space to exist between the valve housing and the surrounding aortic wall. Without this clear space, there is a risk for obstruction of the coronary arteries by the valve housing that could compromise blood flow into the coronary arteries.

In an embodiment, portion 105 angles outwards from portion 104 at an angle 106. It is this angle 106 that leads to clearance around the valve housing which avoids obstruction of the coronary arteries.

Thus, an embodiment such as the embodiment of FIG. 1 has at least the following novel collection of features: (1) cuff 120 has a downward sloping and outwardly angled (see angle 106) portion 105, (2) cuff 120 has scalloped portions to better match the cuff to fibrous tissue of the annulus, (3) cuff 120 extends below the bottom most edge of the valve body 101, and/or (4) the system 100 is a suprannular device.

An embodiment includes a prosthetic heart valve comprising: a valve body having a longitudinal axis (e.g., 150) and outlet and inlet ends; at least one leaflet within the valve body to intermittently seal the valve body and allow blood flow through the valve body from the inlet end to the outlet end; a sewing cuff, surrounding the valve body, having upper (e.g., 104) and lower (e.g., 105) portions; wherein (a) the cuff extends below the inlet end (e.g., 108) but does not extend above the outlet end; (b) the lower portion slopes down and away from the longitudinal axis (e.g., angle 106); (c) the lower portion includes scalloped portions (e.g., 160, 161, 162).

An embodiment provides a bottom half of the lower portion has a maximum width (e.g., 121) that is wider than a maximum width of an upper half of the lower portion.

An embodiment provides the upper and lower portions (e.g., 104, 105) are monolithic with each other. However, in other embodiments they may be stitched together with thread, coupled together with adhesive, and the like.

An embodiment provides a first scalloped portion (e.g., 160) couples to the sewing cuff along a first border (e.g., 160′) and a second scalloped portion (e.g., 162) couples to the sewing cuff along second border (e.g., 162′); and the first scalloped portion rotates about the first border (i.e., rotates “vertically”) and the second scalloped portion rotates about the second border independently of the first scalloped portion rotating about the first border. For example, during vertical rotation towards the top of the valve angle 106 may decrease and during vertical rotation away from the top of the valve angle 106 may increase. In FIG. 3 the portions 160, 161, 162 rotate independently of each other due to voids or recesses 165, 166, 167.

An embodiment provides a first scalloped portion may have an outer edge (e.g., 164) that forms a first arc (e.g., 163) having a first maximum arc length and the second scalloped portion has an outer edge that forms a second arc having a second maximum arc length that is unequal to the first maximum arc length. For example, voids 165, 166, 167 may be spaced equidistant from each other in some embodiments (so arc lengths for the scalloped portions would be equal) but not necessarily so in other embodiments (so arc lengths for the scalloped portions would be unequal).

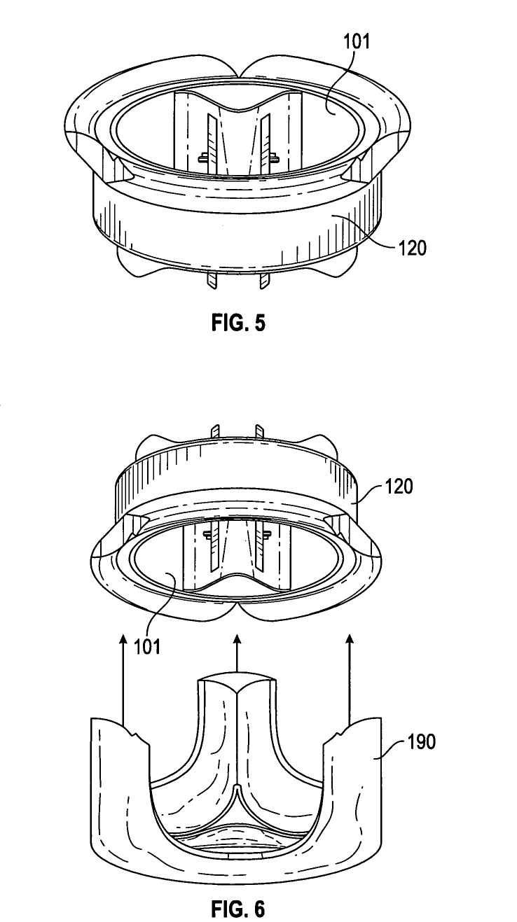

In an embodiment first, second, and third scalloped portions (e.g., 160, 161, 162) collectively have an outer profile that is circular. See e.g., FIG. 5.

In an embodiment the inlet end (e.g., 152) includes a bottom-most edge (e.g., 153) of the valve body (e.g., 101).

An embodiment provides a lower portion that includes a sloping portion that slopes down and away (e.g., angle 106) from the long axis (e.g., axis 150); and a horizontal axis (e.g., axis 168) intersects the valve body and the sloping portion.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A prosthetic heart valve comprising:
a valve body having a longitudinal axis and outlet and inlet ends;
at least one leaflet within the valve body to intermittently seal the valve body and allow blood flow through the valve body from the inlet end to the outlet end;
a sewing cuff, surrounding the valve body, having upper and lower portions;
wherein (a) the cuff includes extends below the inlet end but does not extend above the outlet end; (b) the lower portion slopes down and away from the longitudinal axis; and (c) the lower portion includes scalloped portions.

2. The valve of claim 1, wherein the valve is a supraannular valve.

3. The valve of claim 2, wherein a bottom half of the lower portion has a maximum width that is wider than a maximum width of an upper half of the lower portion.

4. The valve of claim 2, wherein the upper and lower portions are monolithic with each other.

5. The valve of claim 2, wherein the lower portion rotates about the valve body independently from the upper portion.

6. The valve of claim 2, wherein the cuff rotates about the valve body.

7. The valve of claim 6, wherein the valve is an aortic valve.

8. The valve of claim 7, wherein the cuff couples to the valve body via a resistance fit.

9. The valve of claim 2, wherein the lower portion includes:
a first recess separating a first scalloped portion from a second scalloped portion; and
a second recess separating the second scalloped portion from a third scalloped portion.

10. The valve of claim 9, wherein:
the first scalloped portion couples to the cuff along a first border and the second scalloped portion couples to the cuff along second border; and
the first scalloped portion rotates about the first border and the second scalloped portion rotates about the second border independently of the first scalloped portion rotating about the first border.

11. The valve of claim 9, wherein the first scalloped portion has an outer edge that forms a first arc having a first maximum arc length and the second scalloped portion has an outer edge that forms a second arc having a second maximum arc length that is unequal to the first maximum arc length.

12. The valve of claim 9, wherein the first, second, and third scalloped portions collectively have an outer profile that is circular.

13. The valve of claim 2, wherein the inlet end includes a bottom-most edge of the valve body.

14. The valve of claim 2, wherein:
the lower portion overlaps the upper portion; and
a horizontal axis intersects the upper and lower portions.

15. The valve of claim 1, wherein:
the lower portion includes a sloping portion that slopes down and away from the longitudinal axis; and
a horizontal axis, orthogonal to the vertical axis, intersects the valve body and the sloping portion.

16. The valve of claim 1, wherein the valve is entirely synthetic and includes no animal tissue.

17. A prosthetic heart valve comprising:
a supraannular aortic valve body having a longitudinal axis and outlet and inlet ends;
a leaflet in the valve body to allow blood flow from the inlet end to the outlet end; and
a sewing cuff, surrounding the valve body, having upper and lower portions;
wherein (a) the cuff extends below the inlet end, (b) the lower portion slopes down and away from the longitudinal axis, and (c) the lower portion includes first and second voids separating the lower portion into at least first, second, and third portions that each rotate vertically independently of each other.

18. The valve of claim 17, wherein the cuff rotates about the longitudinal axis.

19. The valve of claim 18, wherein a bottom half of the lower portion has a maximum width that is wider than a maximum width of an upper half of the lower portion.

20. The valve of claim 18, wherein the cuff couples to the valve body via a resistance fit
